(19) United States
(12) Patent Application Publication
(10) Pub. No.: US 2011/0009359 A1
(43) Pub. Date: Jan. 13, 2011

(54) USE OF NON-DIGESTIBLE CARBOHYDRATES FOR IMPROVING INTESTINAL MICROBIOTA

(75) Inventors: Kaouther Ben Amor, Utrecht (NL); Jan Knol, Wageningen (NL); Willem Ferdinand Nieuwenhuizen, Bunnik (NL); Eline Marleen Van Der Beek, Wageningen (NL); Gelske Speelmans, Wageningen (NL)

Correspondence Address: FOLEY AND LARDNER LLP;SUITE 500, 3000 K STREET NW, WASHINGTON, DC, 20007, US

(73) Assignee: N.V. Nutricia

(21) Appl. No.: 12/809,430

(22) PCT Filed: Dec. 19, 2008
(86) PCT No.: PCT/NL08/50824

§ 371 Date: Sep. 15, 2010

Related U.S. Application Data
(60) Provisional application No. 61/040,788, filed on Mar. 31, 2008.

(30) Foreign Application Priority Data
Dec. 21, 2007 (EP) 07124007.1
Mar. 31, 2008 (EP) 08153775.5

(51) Int. Cl. A61K 31/733; A61P 3/10; A61P 9/00; A61P 1/16; A61P 3/06

(52) U.S. Cl. 514/54

(57) ABSTRACT

A composition comprising at least two non-digestible carbohydrates for providing and/or maintaining an optimal intestinal microbiota is provided. The composition is especially suitable for infant nutrition.

### FIELD OF THE INVENTION

The present invention is in the field of nutritional compositions for restoring or maintaining a well balanced intestinal microbiota. The composition is especially suitable for infants and toddlers.

### BACKGROUND OF THE INVENTION

At birth infants are devoid of an intestinal microbiota. After birth the large intestine is quickly colonised and its microbiota rapidly develops and increases. The intestinal microbiota of breast-fed infants is primarily composed of species belonging to the genus Bifidobacterium and other lactic acid producing bacteria. The microbiota of formula-fed infants is more diverse and contains in general more species belonging to Bacteroides, Clostridium and Enterobacteriaceae. This difference in microbiota composition is alleged to result in many beneficial effects regarding the immune system, the gastro-intestinal barrier function and anti-pathogenic function, resulting in less gastro-intestinal infections in terms of both incidence and duration, less atopic diseases such as food allergy, atopic dermatitis, allergy induced asthma, and less constipation in breast-fed infants.

WO 2006/091103 discloses a composition comprising probiotics and two non-digestible saccharides for the use against several disorders. WO 00/08948 discloses mixtures of non-digestible oligosaccharides and polysaccharides in order to improve the intestinal microbiota.

WO 2007/073194 discloses the use of compositions comprising phospholipids, sphingolipids and cholesterol and optionally non-digestible carbohydrates for use in prevention of obesity and/or diabetes.

### SUMMARY OF THE INVENTION

The present invention is based on an unexpected advantageous effect of a composition comprising at least two non-digestible oligosaccharides on the composition and/or activity of the intestinal microbiota. It was unexpectedly found that administration of a nutritional composition comprising at least two different non-digestible carbohydrates resulted in a decreased ratio of Firmicutes/Bacteroidetes and/or a decreased ratio Clostridium/Bacteroides.

The inventors recognized that the composition of the microbiota formed during infancy affects the composition of the microbiota later in life. Differences in microbiota development during infancy are considered to result in differences in the composition of intestinal microbiota in children of at least seven years old. The development of a well-balanced microbiota during infancy therefore has health consequences extending beyond infancy, into childhood, adolescence and/or adulthood.

Hence the use of compositions comprising at least two non-digestible carbohydrates, will, when administered early in life to infants and/or toddlers with an age below 36 months of age, beneficially affect the prevention of disorders, associated with a imbalanced intestinal microbiotia, for periods extending beyond the period when this composition is administered, i.e. above 36 months of age, such as childhood, adolescence and/or adulthood.

It is thus considered that a well-balanced microflora, especially a microflora with a low ratio of Firmicutes/Bacteroidetes or Clostridia/Bacteroides, affects the fat storage in adipocytes and/or has a preventive and/or therapeutic effect on obesity. Therefore, the present composition is particularly suitable for treating and/or preventing obesity. Since adipocytes, especially visceral adipocytes, are formed and proliferate during infancy, the present composition is especially suitable for administration to human subjects with an age below 36 months for prevention of obesity when said human subject has reached an age above 36 month of age.

### DETAILED DESCRIPTION OF THE INVENTION

The present invention concerns a method for decreasing the ratio of Firmicutes/Bacteroidetes and/or Clostridia/Bacteroides in the intestinal microbiota in a human subject towards the ratio found in healthy lean subjects and/or towards the ratio found in breast-fed infants, and/or with respect to the ratio present in the human subject prior to ingestion of the present composition, and/or to a control group not taking the present composition said method comprising administering a composition comprising at least two non-digestible carbohydrate selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides to said human subject.

In other words, the present invention concerns the use of a nutritional composition comprising at least two non-digestible carbohydrates selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides for the manufacture of a composition for decreasing the ratio Firmicutes/Bacteroidetes and/or Clostridia/Bacteroides in the intestinal microbiota in a human subject towards the ratio found in healthy lean subjects and/or towards the ratio found in breast-fed infants and/or with respect to the ratio present in the human subject prior to ingestion of the composition, and/or to a control group not taking the composition.

The present invention can also be worded as a nutritional composition comprising at least two non-digestible carbohydrates selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides for decreasing the ratio of Firmicutes/Bacteroidetes and/or Clostridia/Bacteroides in the intestinal microbiota in a human subject towards the ratio found in healthy lean subjects and/or towards the ratio found in breast-fed infants and/or with respect to the ratio present in the human subject prior to ingestion of the composition, and/or to a control group not taking the composition.

Non-digestible carbohydrates

The present composition comprises at least two different non-digestible carbohydrates.

Non-digestible carbohydrates improve the microbiota. Non-digestible carbohydrates stimulate the growth and/or activity of bifidobacteria. As is shown in example 1, administration of at least two non-digestible carbohydrates was found to advantageously decrease the ratio of Firmicutes/Bacteroidetes, i.e. Clostridia/Bacteroides.

Preferably, the present composition comprises non-digestible carbohydrate with a DP between 2 and 250, more preferably 2 to 60. The non-digestible carbohydrate are at least two selected from the group consisting of fructo-oligosaccharides, galacto-oligosaccharides, gluco-oligosaccharides, arabino-oligosaccharides, mannan-oligosaccharides, xylo- oligosaccharides, fuco-oligosaccharides, arabinogalacto-oligosaccharides (including gum acacia), glucomanno-oligosaccharides, galactomanno-oligosaccharides sialic acid comprising oligosaccharides and uronic acid oligosaccharides. Preferably the present composition comprises fructo-oligosaccharides, galacto-oligosaccharides and/or galacturonic acid oligosaccharides, more preferably galacto-oligosaccharides, most preferably beta-galacto-oligosaccharides. The group of fructo-oligosaccharides includes inulin, the group of galacto-oligosaccharides includes transgalacto- oligosaccharides or beta-galacto-oligosaccharides, the group of gluco-oligosaccharides includes gentio-, nigero- and cyclodextrin-oligosaccharides and polydextrose, the group of arabinogalacto-oligosaccharides includes gum acacia, the group of galactomanno-oligosaccharides includes partially hydrolysed guar gum.

Preferably the composition comprises galacto-oligosaccharides. The galacto-oligosaccharide is preferably selected from the group consisting of beta-galacto-oligosaccharides, lacto-N-tetraose (LNT), lacto-N-neotetraose (neo-LNT), fucosyl-lactose, fucosylated LNT and fucosylated neo-LNT. In a particularly preferred embodiment the present composition comprises beta-galacto-oligosaccharides. Beta-galacto-oligosaccharides as used in the present invention refers to oligosaccharides composed of over 50%, preferably over 65% galactose units based on monomeric subunits, with a degree of polymerization (DP) of 2 to 20, in which at least 50%, more preferably at least 75%, even more preferably at least 90%, of the galactose units are linked together via a beta-glycosidic linkage, preferably a beta-1,4 glycosidic linkage. The average DP is preferably of 3 to 6. A glucose unit may be present at the reducing end of the chain of galactose units. Beta-galacto-oligosaccharides are sometimes also referred to as transgalacto-oligosacchariodes (TOS). A suitable source of beta-galacto-ologosaccharides is Vivinal®GOS (commercially available from Borculo Domo Ingredients, Zwolle, Netherlands). Other suitable sources are Oligomate (Yakult), Cupoligo, (Nissin) and Bi2muno (Classado).

Preferably the composition comprises fructo-oligosaccharides. Fructo-oligosaccharide as used in the present invention refers to carbohydrates composed of over 50%, preferably over 65% fructose units based on monomeric subunits, in which at least 50%, more preferably at least 75%, even more preferably at least 90%, of the fructose units are linked together via a beta-glycosidic linkage, preferably a beta-2,1 glycosidic linkage. A glucose unit may be present at the reducing end of the chain of galactose units. Preferably the fructo-oligosaccharide has a DP or average DP of 2 to 250, more preferably 2 to 100, even more preferably 10 to 60. Fructo-oligosaccaride comprises levan, hydrolysed levan, inulin, hydrolysed inulin, and synthesised fructo-oligosaccharides. Preferably the composition comprises short chain fructo-oligosaccharides with an average degree of polymerization (DP) of 3 to 6, more preferably hydrolysed inulin or synthetic fructo-oligosaccharide. Preferably the composition comprises long chain fructo-oligosaccharides with an average DP above 20, such as RaftilinHP. Preferably the composition comprises both short chain and long chain fructo-oligosaccharides. Fructo-oligosaccharide suitable for use in the compositions is also readily commercially available, e.g. RaftilineHP (Orafti).

The present composition preferably comprises uronic acid oligosaccharides, more preferably galacturonic acid oligosaccharides. The term galacturonic acid oligosaccharide as used in the present invention refers to an oligosaccharide wherein at least 50% of the monosaccharide units present in the oligosaccharide is galacturonic acid. The galacturonic acid oligosaccharides used in the invention are preferably prepared from degradation of pectin, pectate, and/or polygalacturonic acid. Preferably the degraded pectin is prepared by hydrolysis and/or beta-elimination of fruit and/or vegetable pectins, more preferably apple, citrus and/or sugar beet pectin, even more preferably apple, citrus and/or sugar beet pectin degraded by at least one lyase. In a preferred embodiment, at least one of the terminal galacturonic acid units of the galacturonic acid oligosaccharide has a double bond. The double bond effectively protects against attachment of pathogenic bacteria to intestinal epithelial cells. Preferably one of the terminal galacturonic acid units comprises a C₄-C₅ double bond. The galacturonic acid oligosaccharide can be derivatised. The galacturonic acid oligosaccharide may be methoxylated and/or amidated. In one embodiment the galacturonic acid oligosaccharides are characterised by a degree of methoxylation above 20%, preferably above 50% even more preferably above 70%.

Thus, in one embodiment the composition for use according to the present invention comprises at least beta-galacto-oligosaccharides. In one embodiment the composition for use according to the present invention comprises at least short chain fructo-oligosaccharides and/or long chain fructo-oligosaccharides, preferably long chain fructo-oligosaccharides. In one embodiment the composition for use according to the present invention comprises at least uronic acid oligosaccharides. In one embodiment the composition for use according to the present invention comprises at least beta-galacto-oligosaccharides and at least short chain fructo-oligosaccharides or long chain fructo-oligosaccharides or both. In one embodiment the composition for use according to the present invention comprises at least beta-galacto-oligosaccharides and at least uronic acid oligosaccharides. In one embodiment the composition for use according to the present invention comprises at least short chain fructo-oligosaccharides and uronic acid oligosaccharides or long chain fructo-oligosaccharides and uronic acid oligosaccharides. In one embodiment the composition for use according to the present invention comprises at least beta-galacto-oligosaccharides and short chain fructo-oligosaccharides and uronic acid oligosaccharides or at least beta-galacto-oligosaccharides and long chain fructo-oligosaccharides and uronic acid oligosaccharides.

In a preferred embodiment the composition comprises a mixture of inulin and fructo-oligosaccharides. In a preferred embodiment the composition comprises a mixture of galacto-oligosaccharides and fructo-oligosaccharides selected from the group consisting of short chain fructo-oligosaccharides and inulin, more preferably inulin. A mixture of at least two different non-digestible carbohydrates advantageously stimulates the beneficial bacteria of the intestinal microbiota to a greater extent than based on the single non-digestible carbohydrates. Preferably the weight ratio between the mixture of two different non-digestible carbohydrates, preferably galacto-oligosaccharides and fructo-oligosaccharide, is between 20 and 0.05, more preferably between 20 and 1. Galacto-oligosaccharides are more reminiscent to the human milk oligosaccharides. Preferably the present composition comprises galacto-oligosaccharides with a DP of 2-10 and/or fructo-oligosaccharides with a DP of 2-60. This combination was found to synergistically increase bifidobacteria and lactobacilli. Preferably the composition comprises beta-galacto-oligosaccharide, fructo-oligosaccharide and a pectin degradation product. The presence of these three non-digestible oligosaccharides even further stimulates the bifidobacteria, while decreasing bacteroides and clostridia compared to galacto-oligosaccharides with fructo-oligosaccharides and even further decreases the ratio of Firmicutes/Bacteroidetes and/or the ratio of Clostridium/Bacteroides. The weight ratio transgalacto-oligosaccharide : fructo-oligosaccharide : pectin degradation product is preferably (20 to 2):1:(1 to 20), more preferably (12 to 7):1:(1 to 3).

Preferably, the composition comprises of 80 mg to 2 g non-digestible oligosaccharides per 100 ml, more preferably 150 mg to 1.50 g, even more preferably 300 mg to 1 g.

Based on dry weight, the composition preferably comprises 0.25 wt. % to 20 wt. %, more preferably 0.5 wt. % to 10 wt. %, even more preferably 1.5 wt. % to 7.5 wt. %. A lower amount of non-digestible oligosaccharides will be less effective in stimulating the beneficial bacteria in the microbiota, whereas a too high amount will result in side-effects of bloating and abdominal discomfort.

Lipids Herein LA refers to linoleic acid (18:2 n6); ALA refers to alpha-linolenic acid (18:3 n3); LC-PUFA refers to long chain polyunsaturated fatty acids and/or acyl chains comprising at least 20 carbon atoms in the fatty acyl chain and with 2 or more unsaturated bonds; DHA refers to docosahexaenoic acid (22:6, n3); EPA refers to eicosapentaenoic acid (20:5 n3); ARA refers to arachidonic acid (20:4 n6); DPA refers to docosapentaenoic acid (22:5 n3).

LA preferably is present in a sufficient amount in order to promote a healthy growth and development, yet in an amount as low as possible to prevent occurrence of obesity later in life. The composition therefore preferably comprises less than 15 wt. % LA based on total fatty acids, preferably between 5 and 14.5 wt. %, more preferably between 6 and 12 wt. %. Preferably ALA is present in a sufficient amount to promote a healthy growth and development. The present composition therefore preferably comprises at least 1.0 wt. % based on total fatty acids. Preferably the composition comprises at least 1.6 wt. % ALA based on total fatty acids, more preferably at least 2.0 wt. %. Preferably the composition comprises less than 10 wt. % ALA, more preferably less than 5.0 wt. % based on total fatty acids. The weight ratio LA/ALA should be well balanced in order to prevent obesity, especially central obesity, while at the same time ensuring a normal growth and development. Therefore, the present composition preferably comprises a weight ratio of

LA/ALA between 2 and 15, more preferably between 2 and 7, more preferably between 3 and 6, even more preferably between 4 and 5.5, even more preferably between 4 and 5.

Preferably the present composition comprises LC-PUFA, since LC-PUFA reduce obesity later in life, more preferably central obesity. More preferably, the present composition comprises n-3 LC-PUFA, even more preferably EPA, DPA and/or DHA, even more preferably DHA. It was found that these n-3 LC-PUFA decrease obesity. Since a low concentration of DHA, DPA and/or EPA is already effective and normal growth and development are important, the content of n-3 LC-PUFA in the present composition, preferably does not exceed 15 wt. % of the total fatty acid content, preferably does not exceed 10 wt. %, even more preferably does not exceed 5 wt. %. Preferably the present composition comprises at least 0.2 wt. %, preferably at least 0.5 wt. %, more preferably at least 0.75 wt. %, n-3 LC-PUFA of the total fatty acid content. The weight ratio n-6 LC-PUFA/n-3 LC-PUFA in the present composition is preferably low in order to prevent obesity later in life. Preferably the composition comprises a weight ratio of n-6 LC-PUFA/n-3 LC-PUFA below 1.5, more preferably below 1.0, even more preferably below 0.6. LC-PUFA have the further advantage that they facilitate the adhesion of beneficial bacteria, thereby further improving the microbiota and/or maintaining a well balanced microbiota.

The present composition preferably comprises sphingophospholipids and/or one of its degradation products. The term sphingophospholipid as in the present invention relates to a type of sphingolipid consisting of a sphingoid base bonded to one fatty acid via an amide bond and, via an oxygen, to one polar headgroup comprising a phosphate (phosphate, phosphocholine, phosphoserine, phosphoethanolamine, phosphoglycerol, or phosphoinositol). In another embodiment of the present invention a degradation product derived from sphingophospholipid, particularly ceramides, lysosphingophospholipid, sphingoid phosphate and/or the free sphingoid base, is preferably present in the composition. Sphingoid bases in the present invention are preferably 2-amino-1,3-dihydroxy-alkanes or alkenes with (2S.3R)-erythro stereochemistry, with a chain length of 14 atoms or higher. Sphingoid bases may comprise three instead of two hydroxyl groups. The aliphatic chains may be mono- or poly-unsaturated (in the cis- or trans-configuration) or saturated. The aliphatic chain may be branched. Sphingoid bases include sphingosine, sphinganine, phytosphingosine and dihydrophytosphingosine. Preferably the composition comprises sphingomyelin. Sphingomyelin is ceramide phosphorylcholine. Ceramide consists of a sphingoid base linked to a fatty acid via an amide bond.

Upon oral consumption, sphingophospholipids and/or degradation products thereof, in particular lysosphingolipid, ceramide, sphingoid phosphate and/or sphingoid base, has a reducing effect on unfavourable bacteria in the intestinal microbiota, such as firmicutes, especially clostridia. Having both a stimulator of beneficial bacteria and an inhibitor of unfavourable bacteria present will improve the composition of the microbiotia and/or keep the microbiota well balanced in a much more effective way.

Preferably, the composition comprises 10 to 200 mg sphingomyelin per 100 ml, more preferably 20 mg to 150 mg, even more preferably 60 mg to 100 mg. Based on fat, the composition preferably comprises 0.25 wt. % to 5.5 wt. %, more preferably 0.5 wt. % to 4 wt. %, even more preferably 1.5 wt. % to 3 wt. %. Based on dry weight, the composition preferably comprises 0.07 wt. % to 1.5 wt. %, more preferably 0.15 wt. % to 1.1 wt. %, even more preferably 0.5 wt. % to 0.8 wt. %. A lower amount will be less effective, whereas a higher amount will leave less room for the advantageous essential and long-chain fatty acids. A suitable source of sphingophospholipid is milk fat, more suitable butter milk fat or butter serum fat, egg yolk and/or soy lecithin.

Preferably the weight ratio between non-digestible oligosaccharides and sphingomyelin is between 0.4 and 200, more preferably between 1 and 75, even more preferably between 3 and 15. Having this ratio present in the composition ensures a balance between the stimulating effect on beneficial bacteria in the microbiota and a reducing effect on unfavourable bacteria in the microbiota.

Preferably the present composition comprises cholesterol and/or cholesterylester. The simultaneous presence of sphingophospholipid and cholesterol and/or cholesterylester in the composition delays the digestion of the sphingophospholipid in the small intestine, thereby increasing the amounts of sphingophospholipid which reaches the large intestine and hence the activity of sphingophospholipid and/or degradation products thereof against unfavourable bacteria in the intestinal microbiota. The present composition preferably comprises at least 0.005 wt. % cholesterol and/or cholesterylester based on total fat, more preferably at least 0.01 wt. %, more preferably at least 0.05 wt. %, even more preferably at least 0.1 wt. %. Preferably the amount of cholesterol and/or cholesteryester does not exceed 10 wt. % based on total lipid, more preferably does not exceed 5 wt. %, even more preferably does not exceed 1 wt. % of total lipid. Most preferably the amount of cholesterol is 0.5 to 0.7 wt. % based on total lipid.

Nutritional Compositions

The present composition is preferably orally administered. The present composition is preferably a nutritional formula, preferably an infant formula. The present composition comprising at least two non-digestible carbohydrates is not human milk.

The present composition can advantageously be applied as a complete nutrition for infants. The present composition preferably comprises lipid, protein and digestible carbohydrate and is preferably administered in liquid form. The present invention includes dry food (e.g. powders) which is accompanied with instructions as to mix said dry food mixture with a suitable liquid (e.g. water).

The present invention advantageously provides a composition wherein the lipid provides 5% to 50% of the total calories. The present invention advantageously provides a composition wherein the protein provides 5% to 50% of the total calories. The present invention advantageously provides a composition wherein the digestible carbohydrate provides 15% to 90% of the total calories. The present invention advantageously provides a composition wherein the lipid provides 5% to 50% of the total calories, the protein provides 5% to 50% of the total calories, and the digestible carbohydrate provides 15% to 90% of the total calories. Preferably, in the present composition the lipid provides 35% to 50% of the total calories, the protein provides 7.5% to 12.5% of the total calories, and the digestible carbohydrate provides 40% to 55% of the total calories. For calculation of the percentage of total calories for the protein, the total of energy provided by the proteins, peptides and amino acids needs to be taken into account. Total calories relates to the sum of the calories provided by the lipid, the digestible carbohydrate and the protein.

The protein used in the nutritional preparation is preferably selected from the group consisting of non-human animal proteins (preferably milk proteins), vegetable proteins (preferably soy protein and/or rice protein), free amino acids and mixtures thereof. The present composition preferably contains casein, whey, hydrolysed casein and/or hydrolysed whey protein. Preferably the protein comprises intact proteins, more preferably intact bovine whey proteins and/or intact bovine casein proteins. As the present composition is suitably used to reduce the allergic reaction in an infant, the protein of is preferably selected from the group consisting of hydrolysed milk protein.

The present composition preferably contains digestible carbohydrate selected from the group consisting of sucrose, lactose, glucose, fructose, corn syrup solids, starch and maltodextrins, more preferably lactose.

Preferably the present composition comprises nucleotides and/or nucleosides, more preferably nucleotides. Preferably, the composition comprises cytidine 5′-monophospate, uridine 5 ‘-monophospate, adenosine 5’-monophospate, guanosine 5′-monophospate, and/or inosine 5′-monophospate, more preferably cytidine 5′-monophospate, uridine 5′-monophospate, adenosine 5′-monophospate, guanosine 5′-monophospate, and inosine 5′-monophospate. Preferably the composition comprises 5 to 100 mg, more preferably 5 to 50 mg, most preferably 10 to 50 mg nucleotides and/or nucleosides per 100 g dry weight of the composition. The presence of nucleotides and/or nucleotides advantageously increases the bacteroides, thereby decreasing the ratio Firmicutes/Bacteroidetes and/or Clostridium/Bacteroides.

The nucleotides and/or nucleosides are deemed to act synergistically with the other ingredients of the present composition.

Stool irregularities, e.g. hard stools, insufficient stool volume, diarrhoea, is an important problem in babies. It was found that stool problems may be reduced by administering the present composition in a liquid form with an osmolality between 50 and 500 mOsm/kg, more preferably between 100 and 400 mOsm/kg. The reduced stool irregularities enhance the colonization and development of a healthy intestinal microbiota.

In view of the above, it is also important that the liquid food does not have an excessive caloric density, however still provides sufficient calories to feed the subject. Hence, the liquid food preferably has a caloric density between 0.1 and 2.5 kcal/ml, even more preferably a caloric density of between 0.5 and 1.5 kcal/ml, most preferably between 0.6 and 0.8 kcal/ml.

Applications

The present composition comprising at least two non-digestible carbohydrate is particularly suitable for use in a human subject having an age below 36 months for use in decreasing the ratio Firmicutes/Bacteroidetes and/or Clostridium/Bacteroides in the intestinal microbiota. The present composition is particularly suitable for use in a human subject having an age below 36 month for use in decreasing the ratio Firmicutes/Bacteroidetes and/or Clostridium/Bacteroides in the intestinal microbiota when said human subject has reached an age above 36 months.

Decreasing the ratio Firmicutes/Bacteroidetes and/or Clostridium/Bacteroides in the intestinal microbiota in the present human subjects below 36 months of age relates to decreasing the ratio Firmicutes/Bacteroidetes and/or Clostridium/Bacteroides in the intestinal microbiota towards the ratio found in the intestinal microbiota of healthy human milk fed human subjects with an age below 36 months and/or with respect to the ratio present in the human subject prior to ingestion of the present composition, and/or to a control group not taking the present composition.

The use in a human subject having an age below 36 months for use in decreasing the ratio Firmicutes/Bacteroidetes and/or Clostridium/Bacteroides in the intestinal microbiota when said human subject has reached an age above 36 months, relates to decreasing the ratio Firmicutes/Bacteroidetes and/or Clostridium/Bacteroides in the intestinal microbiota towards the ratio found in the intestinal microbiota of healthy subjects of the same age which have been human milk fed in the past when they were of an age below 36 months and/or with respect to the ratio present in the human subject prior to ingestion of the present composition, and/or to a control group not taking the present composition.

The present composition comprising at least two non-digestible carbohydrates is particularly suitable for use in a human subject having an age above 36 months for use in decreasing the ratio Firmicutes/Bacteroidetes and/or Clostridium/Bacteroides in the intestinal microbiota. Decreasing the ratio Firmicutes/Bacteroidetes and/or Clostridium/Bacteroides in the intestinal microbiota in human subjects above 36 months of age relates to decreasing the ratio Firmicutes/Bacteroidetes and/or Clostridium/Bacteroides in the intestinal microbiota towards the ratio found in the intestinal microbiota of healthy human subjects of normal weight and/or with respect to the ratio present in the human subject prior to ingestion of the present composition, and/or to a control group not taking the present composition.

The present composition comprising at least two non-digestible carbohydrates is particularly suitable for use in an obese human subject for use in decreasing the ratio Firmicutes/Bacteroidetes and/or Clostridium/Bacteroides in the intestinal microbiota. Decreasing the ratio Firmicutes/Bacteroidetes and/or ClostridiumBacteroides in the intestinal microbiota in obese human subjects relates to decreasing the ratio Firmicutes/Bacteroidetes and/or Clostridium/Bacteroides in the intestinal microbiota towards the ratio found in the intestinal microbiota of healthy human subjects with normal weight and/or with respect to the ratio present in the human subject prior to ingestion of the present composition, and/or to a control group not taking the present composition.

Preferably the ratio Firmicutes/Bacteroidetes and/or Clostridium/Bacteroides is decreased at least 25%, more preferably at least 33%, even more preferably at least 40%. Preferably the ratio Firmicutes/Bacteroidetes and/or Clostridium/Bacteroides is decreased compared to ratio of Firmicutes/Bacteroidetes and/or Clostridium/Bacteroides in a human subject towards the ratio found in healthy lean human subjects and/or in breast-fed infants and/or with respect to the ratio present in the human subject prior to ingestion of the present composition, or compared to a control group not taking the present composition.

The intestinal microbiota relates in particular to the microbiota composition of the colon.

The composition of the intestinal microbiota can be determined on the level phylum, class, family, genus and/or species by methods known in the art, including FISH, real time PCR and micro-arrays, using specific probes and/or primers known in the art.

The present composition is especially advantageous for infants born in the hospital, since they are at risk of being colonised by unfavourable bacteria deriving from the hospital environment. The present composition is even more advantageous for infants delivered via caesarean section, since these infants lack a proper inoculation with beneficial bacteria deriving from the mother, while at the same time they are at risk of being colonised by unfavourable bacteria deriving from the hospital environment.

Since administration of the present composition to human subjects below 36 months of age has the advantageous effects on microbiota as described above, the present composition is preferably subsequently used for the prevention and/or treatment of obesity and/or adiposity, more preferably visceral obesity, more preferably when said human subject has reached an age above 36 months. Subsequently the present composition preferably is used to prevent secondary disorders resulting from visceral obesity, such as type 2 diabetes, fasting hyperglycaemia, insulin resistance, visceral adiposity, hyperinsulinemia, hypertension, cardiovascular disease, cerebrovascular disease, artherosclerose, dyslipidaemia, hyperuricaemia, fatty liver, osteoarthritis and sleep apnoea when said human subject has reached an age above 36 months. In one embodiment obesity, adiposity and visceral obesity, refer to said conditions as the consequence of unbalanced ratio of Firmicutes/Bacteroidetes and/or Clostridium/Bacteroides in the intestinal microbiota.

The present invention aims to decreasing the ratio Firmicutes/Bacteroidetes and/or Clostridium/Bacteroides in the intestinal microbiota in human subjects preferably with an age above 36 months, preferably an age above 8 years, more preferably an age above 15 years, most preferably above 18 years.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

Infants of 9-10 months of age were given 500 ml per day of a milk formula comprising 1.2 g beta-galacto-oligosaccharides plus long chain inulin for 1 month (group A). A control group received milk formula without beta-galacto-oligosaccharides and long chain inulin (group B). In total 138 children entered the study. The faecal flora was examined before and after this period by FISH analysis.

The faecal flora was examined at 12 and 13 months by FISH analysis as described previously (Haarman et al. Appl. Environ.Microbiol. 2005. May; 71(5): 2318-24). A set of 7 probes was used to enumerate the major fecal bacterial groups of the children. These were (i) Bif164 to detect the genus Bifidobacterium, (ii) Erec482 for Eubacteria rectale/Clostridium coccoides (iii) Bac303 for Bacteroides/Prevotella group, (iv) Chis150 to detect the Clostridium histolyticum group with C.difficile as the predominant species, (v) Clit135 to detect C. lituseburense group with C. perfringens as the predominant species (vi) Lab158 for lactobacilli and enterococci, and (vi) Ec1531 for E. coli and related species. The Eub338 probe was used as a positive control since it targets almost all bacteria. For more details about the used probes, see Harmsen, HJM et al. 2002, AEM. 68: 2982-2990, Franks A H et al. 1998. AEM. 64: 3336-3334. For determining Firmicutes the sum of Erec482, Chis150, Cli135 and Lab158 was taken. For determining Clostridium the sum of the Chis150 and Clit was taken. For determining Bacteroidetes or Bacteroides the Bac303 probe was used.

After the intervention time, the ratio of Bifidobacterium/total bacteria was significantly higher in group A than in group B. Also the ratio Clostridium/total bacteria was significantly lower in group A than in Group B. The ratio Firmicutes/Bacteroidetes was 42 in group A and 39 in group B at the start and was decreased to 32 in group B and to 16 in group A after the 1 month intervention. Also the ratio Clostridium/Bacteroides decreased. Also the ratio Eubacterium rectale+Clostridium coccoides/total bacteria significantly decreased. Also the ratio Enterobacterium/total bacteria decreased. These data are also shown in table 1.

TABLE 1
Percentage of different bacterial groups in children's faeces of children receiving
a formula with beta-galacto-oligosaccharides and long chain inulin (A) or a control
formula (B) as a mean % of total bacteria (Eub338probe) with standard error at 12 and 13
months.

| probe | A before | B before | A after | B after |
|---|---|---|---|---|
| Erec482 | 18.9 (2.1) | 31.1 (2.3) | 22.9 (2.5) | 29.4 (2.8) |
| Chis150 + Clit135 | 2.9 (0.5) | 5.6 (0.8) | 3.9 (0.5) | 4.7 (0.6) |
| Lab158 | 1.5 (0.5) | 1.6 (0.8) | 1.9 (0.9) | 1.7 (0.8) |
| Firmicutes | 23.3 | 38.3 | 27.0 | 35.8 |
| Bac303 | 0.56 (0.29) | 0.98 (0.64) | 1.65 (0.48) | 1.13 (0.35) |
| ratio C/B_{^{a}} | 5.2 | 5.7 | 2.4 | 4.2 |
| ratio F/B_{^{b}} | 42 | 39 | 16 | 32 |
| Bif164 | 18.9 (2.1) | 31.1 (2.3) | 41.1 (3.4) | 31.0 (3.5) |
| Ec1531 | 0.14 (0.09) | 0.17 (0.35) | 0.32 (0.14) | 0.60 (0.43) |

_{^{a}}C/B is ratio Clostridium/Bacteroides, (Chis150 + Clit135)/Bac303
_{^{b}}F/B is ratio Firmicutes/Bacteroidetes, (Erec482 + Chis150 + Clit135 + Lab158)/Bac303.

### Example 2

Pooled fresh faeces obtained from 4-5 healthy adults was used to perform fermentation studies as described in Appendix I of WO 2004/000340. The experiments were performed in duplo. The faeces, diluted 1:5 into McBain and Farlane medium, was put into dialysis tubes and suspended in bottles with buffer under anaerobic conditions and was first incubated at 37 ° C. for 16 h for reasons of adaptation before inoculating them with the substrates according to table 1 below at t=0.

The test substrates were added once a day (at t=0, 24, and 48 hours). Buffer was refreshed once a day. A sample was taken at t=0 and t=72. This relatively long period was used since shifts in bacterial populations are relatively slow compared to the shifts in short chain acids formed.

TABLE 2
Additions to the dialysis tube comprising diluted faeces

| Number | Sphingosine | GOS | Inulin |
|---|---|---|---|
| 1 | 0 | 103.7 mg | 11.5 mg |
| 2 | 300 μg | 103.7 mg | 11.5 mg |

As a source of GOS Vivinal GOS (Borculo Domo, The Netherlands was used). As a source of inulin RaftilineHP (Orafti, Belgium) was used.

D-sphingosine was added to the dialysis tubes instead of sphingophospholipid such as sphingomyelin, since degradation of sphingophospholipids in the small intestine gives D-sphingosine. As a source of D-sphingosine D sphingosine from bovine brain (Fluka Sigma Aldrich Chemie) was used.

The sphingosine was dissolved in ethanol (stock 10 mg/ml). 30 μl of a stock of 10 mg/ml was added to the dialysis tube.

The samples were analysed by Fluorescence In Situ Hybridisation (FISH) as described in Franks, A. H., et al., Appl. Environ. Microbiol., 1998. 64(9): p. 3336-3345. For determination of the Bacteroides-Prevotella group the Bac303 probe was used (Manz, W., et al., Microbiology, 1996. 142 (Pt 5): p. 1097-106). For determination of Clostridia the Chis150 and C1it135 probe were used (Franks, A. H., et al., Appl. Environ. Microbiol., 1998. 64(9): p. 3336-3345). For determination of the Eubacterium rectale and Clostridium coccoides group the Erec482 probe was used (Franks, A. H., et al., Appl. Environ. Microbiol., 1998. 64(9): p. 3336-3345). For Bifidobacteria determination the Bif164 probe was used (Langendijk, P., et al., Appl. Environ. Microbiol., 1995. 61(8): p. 3069-3075). For determination of Lactobacilli and Enterococci the Lab158 probe was used (Harmsen, H. J. M., et al., Microbial Ecology in Health and Disease, 1999. Volume 11: p. 3-12). For determination of the enterobacteria the Ec1531 probe was used (Poulsen, L. K., et al., Infect Immun, 1994. 62(11): p. 5191-4). Total number of bacteria was determined by DAPI staining. The ratio Firmicutes/Bacteroidetes was set at the percentage of bacteria detected by the Chis150, Clit135, Erec482 and Lab158 probe, divided by the percentage of bacteria divided by the Bac303 probe. The bacteria detected by the Erec482 group are usually the largest group of Firmicutes in the human intestine.

Results:

The Bac303, Chis150, C1it135, Ec1351, and Lab158 probe all showed signals below the detection limit of FISH, being <10⁶ cells per gram of sample or an relative abundance lower than 1 in 1000 cells (0.1%). In case of the Bac303 the samples were stored under poor conditions, but a faint signal was observed in the samples to which GOS and inulin was added. The FISH results of the Bifl64 and Erec482 probes are shown in Table 3.

TABLE 3
FISH results for Bif164 and Erec482 probes.

| | | Bif164 | Erec482 |
|---|---|---|---|
| Number | Time (h) | % total bacteria (SD) | % total bacteria (SD) |
| | t = 0 | 3.15 | 33.99 |
| 1 | t = 72 | 7.05 | 21.84 |
| 2 | t = 72 | 10.13 | 12.83 |

These results are indicative of an increase in bifidobacteria when non-digestible carbohydrates more particular GOS and/or inulin, are fermented and further increase when sphingosine is present (being the degradation product of sphingophospholipid after digestion in the small intestine) and an even synergistically further increase when both components are present.

These results are indicative that the Clostridia and Eubacteria, being the largest group of intestinal Firmicutes, decrease when non-digestible carbohydrates more particular GOS and/or inulin are fermented. When also sphingosine is present a further improved effect is observed.

These results are indicative for improvement a decreased ratio Firmicutes/Bacteroidetes, and a decreased ratio Clostridia/Bacteroides in the large intestinal microbiota upon consumption of non-digestible carbohydrates, in particular GOS and/or inulin, further improved by the consumption of sphingophospholipids.

### Example 3

### Infant Nutrition

Infant nutrition comprising lipid providing 48% of the total calories, protein providing 8% of the total calories and a digestible carbohydrate providing 44% of the total calories;
(i) the lipid comprising about 1.4 wt. % sphingomyelin and about 4 wt. % cholesterol based on total lipid;
(ii) the digestible carbohydrate comprising 50.9 gram lactose/100 gram powder, and further comprising as non-digestible carbohydrates 5.22 g beta-galacto-oligosaccharides with DP 2-6 and 0.58 g fructo-oligosaccharides with DP 7-60 per 100 g powder;
(iii) the protein comprising cow milk protein, including casein.

The composition comprises vitamins, trace elements and minerals according to EU directives. The label of the package of this infant nutrition indicates that the nutrition aims to decrease the ratio Firmicutes/Bacteroidetes and/or for obtaining a microbiota which will result in obesity reduction.

1.-13. (canceled)

14. A method of decreasing or maintaining the ratio of Firmicutes/Bacteroidetes and/or Clostridium/Bacteroides in a human subject in need thereof, comprising administering to the subject a nutritional composition comprising: (a) fructo-oligosaccharides with an average DP above 20; and (b) galacto-oligosaccharides, wherein the administration of the composition decreases or maintains the ratio toward or at a ration found in healthy lean human subjects or in breast-fed infants.

15. The method according to claim 14, wherein the human subject has an age below 36 months.

16. The method according to claim 15, wherein the ratio remains decreased or is maintained at low levels when the human subject has reached an age above 36 months.

17. The method according to claims 14, wherein the ratio is at least 25% decreased.

18. The method according to claim 14, wherein the composition comprises at least 0.25 wt. % g fructo-oligosaccharides with an average DP above 20 and galacto-oligosaccharides based on dry weight of the composition.

19. The method according to claim 14, wherein the composition further comprises: (c) lipid, wherein the lipid provides 35 to 50% calories based on total calories; (d) digestible carbohydrate, wherein digestible carbohydrate provides 40 to 55% calories based on total calories; and (e) protein, wherein the protein provides 7.5 to 12.5% calories based on total calories of the composition.

20. The method according to claim 14, wherein the composition comprises at least beta-galacto-oligosaccharides.

21. The method according to claim 14, wherein the composition further comprises uronic acid oligosaccharides.

22. A method for the prevention of obesity and/or adiposity, comprising administering to a human subject in need thereof a composition comprising: (a) fructo-oligosaccharides with an average DP above 20; and (b) galacto-oligosaccharides.

23. The method according to claim 22, wherein the composition comprises at least 0.25 wt. % g fructo-oligosaccharides with an average DP above 20 and galacto-oligosaccharides based on dry weight of the composition.

24. The method according to claim 22, wherein the composition further comprises: (c) lipid, wherein the lipid provides 35 to 50% calories based on total calories; (d) digestible carbohydrate, wherein digestible carbohydrate provides 40 to 55% calories based on total calories; and (e) protein, wherein the protein provides 7.5 to 12.5% calories based on total calories of the composition.

25. The method according to claim 22, wherein the composition comprises at least beta-galacto-oligosaccharides.

26. The method according to claim 22, wherein the composition further comprises uronic acid oligosaccharides.

27. The method according to claim 22, wherein the human subject is below 36 months of age and wherein obesity and/or adiposity is prevented when the human subject has reached an age above 36 months.

28. The method according to claim 22, wherein the human subject is susceptible for a disorder selected from the group consisting of type 2 diabetes, fasting hyperglycaemia, insulin resistance, visceral adiposity, hyperinsulinemia, hypertension, cardiovascular disease, cerebrovascular disease, artherosclerose, dyslipidaemia, hyperuricaemia, fatty liver, osteoarthritis and sleep apnoea.
